(19) United States
(12) Patent Application Publication
(10) Pub. No.: US 2020/0038540 A1
(43) Pub. Date: Feb. 6, 2020

(54) ROTATABLE AROMATHERAPY LAMP

(71) Applicant: Huan-Ping Lee, Toufen City (TW)

(72) Inventor: Huan-Ping Lee, Toufen City (TW)

(21) Appl. No.: 16/055,171

(22) Filed: Aug. 6, 2018

(51) Int. Cl. A61L 9/12; F21S 6/00; F21V 29/67

(52) U.S. Cl. CPC A61L 9/122; A61L 2209/12; F21V 29/677; F21S 6/002

(57) ABSTRACT

An aromatherapy lamp includes: a lamp holder, including a base, first, second supports, the base configured with a light bulb seat in combination with a light bulb on the base, the first support configured with a placement portion in combination with a container and positioned above the light bulb, and the second support configured on the top face of the base and having a fulcrum positioned above the container; and a rotatable cover, adapted to cover the base from above, a top end of the rotatable cover configured with a fan and a bottom end thereof formed with an opening, a center of the fan configured with an axial center, the rotatable cover adapted to cover the light bulb seat, first, second supports, and the fulcrum adapted to hold the axial center of the fan, allowing the rotatable cover to be separated from the base.

### (a) TECHNICAL FIELD OF THE INVENTION

The present invention relates to a rotatable aromatherapy lamp, and more particularly to an aromatherapy lamp having a rotatable lamp cover, using the heat energy of a light bulb configured thereon to heat essential oil inside a container, and then using the heat energy of the light bulb to generate rising hot air flow to push a fan configured on the rotatable lamp cover to move, thereby allowing the rotatable lamp cover to be rotated automatically instead of by means of mechanical driving.

### (b) DESCRIPTION OF THE PRIOR ART

To make the environment free of special odor and turbid air so as to increase working efficiency or have a better mental state, there are air cleaners installed indoor or aromatics used to eliminate odors in rooms.

In the case of aromatics, the presence thereof in the interior allows the home to provide a steady stream of fragrance, whether in the room, in the living room or even in the toilet, thereby improving the quality of life at home. In addition, essential oil products are gradually becoming popular with mass consumers; little essential oil is heated to vaporize indoor, not only eliminating indoor odor but making people's mind relaxed.

Conventionally, a liquid or solid aromatic such as perfume, fragrant oil, essence, balm is placed in a container, and the fragrance of the aromatic can be naturally distributed only through the through holes of the container, causing the fragrance not to be able to be distributed to whole indoor space. Similarly, conventional aromatherapy lamps used to accept essential oil are only used to heat the essential oil a little to distribute the vaporized essential oil naturally, causing the fragrance thereof also not to be able to distribute effectively to whole indoor space.

### SUMMARY OF THE INVENTION

The present invention proposes an aromatherapy lamp, including: a lamp holder, including a base, first support and second support, a top face of the base configured with at least one light bulb seat in combination with at least one light bulb configured on a top face of the base, the first support configured with a placement portion in combination with a container and positioned relatively above the light bulb, and the second support configured on the top face of the base and having a fulcrum positioned relatively above the container of the first support; and a rotatable cover, being a hollow body adapted to cover the base of the lamp holder from above, a top end of the rotatable cover configured with a fan and a bottom end thereof formed with an opening, a center of the fan configured with an axial center, the rotatable cover adapted to cover the light bulb seat, first support and second support of the lamp holder, and the fulcrum of the second support adapted to hold the axial center of the fan, allowing the rotatable cover to be separated from the base; whereby, the rotatable cover has a rotation mechanism on the lamp holder.

The present invention configures a fan on the top end of the rotatable cover and the axial center on the center of the fan, thereby using the fulcrum of the second support to hold the fan so that the rotatable cover is separated from the base of the lamp holder, allowing the rotatable cover to has a rotation mechanism. Therefore, when the present invention is put into use, the container is filled with essential oil, and the bulb is used to heat the essential oil inside the container, allowing the essential oil to be vaporized. Furthermore, the heat energy of the bulb can generate rising hot air flow to push the fan configured on the rotatable cover to move, allowing the rotatable cover to be rotated automatically instead of by means of mechanical driving so that the vaporized essential oil can be more spread to indoor space because the rotation of the fan of the rotatable cover increases the flow of airflow.

### BRIEF DESCRIPTION OF THE DRAWINGS

FIG. 1 is a perspective view of a rotatable aromatherapy lamp of the present invention;

FIG. 2 is an exploded view of the rotatable aromatherapy lamp of the present invention;

FIG. 3 is a cross-sectional view of the rotatable aromatherapy lamp of the present invention; and

FIG. 4 is a perspective view of the rotatable aromatherapy lamp of the present invention upon implementation.

### DETAILED DESCRIPTION OF THE PREFERRED EMBODIMENTS

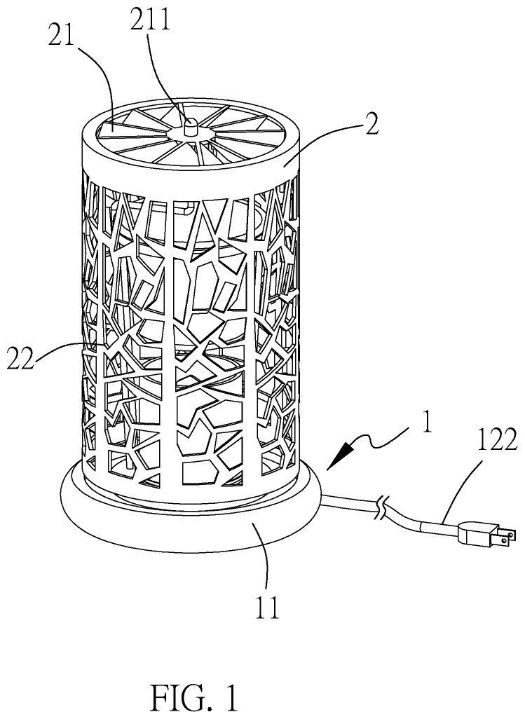

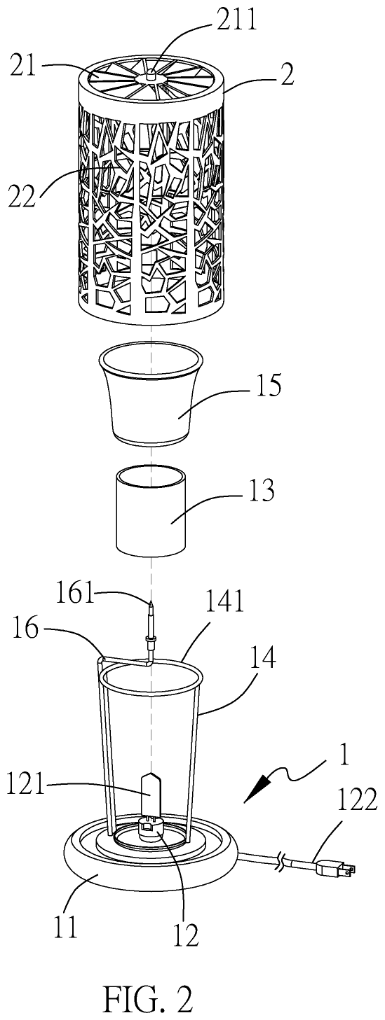

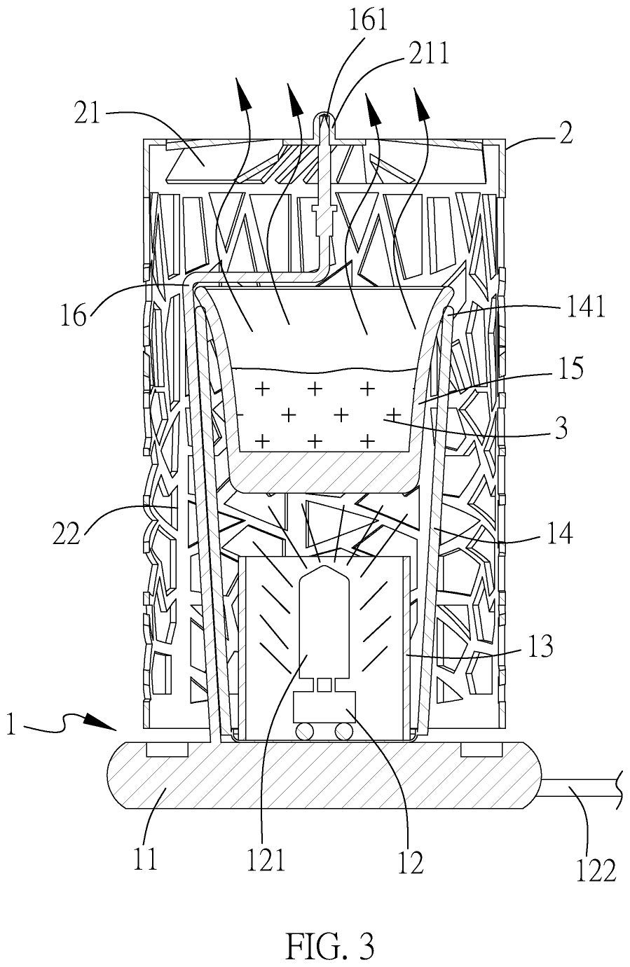

Referring to FIGS. 1 to 4, a rotatable aromatherapy lamp includes a lamp holder 1 and a rotatable cover 2.

The lamp holder 1 includes a base 11, first support 14 and second support 16, where the base 11 has at least one light bulb seat 12 in combination with at least one light bulb 121 in electric connection with an electric wire 122, which is used to plug in a mains socket, allowing the light bulb 12 to be energized to emit light. Furthermore, the base 11 is configured with a hollow tube-shaped diversion shroud 13 adapted to enclose the light bulb seat 12, causing heat flow generated due to the heating of the light bulb can be gathered to flow upward because of the top end opening of the diversion shroud 13. The first support 14 having a placement portion 141 is configured on the top face of the base 11, where the placement portion 141 may be an annular body adapted to be in combination with a container 15, for example, made of glass or ceramics, allowing the container 15 positioned relatively above the light bulb 121 of the light bulb seat 12; the second support 16 is configured on the top face of the base 11, where one end thereof is configured with a tip-shaped fulcrum 161 positioned relatively above the container 15 placed on the placement portion 141. For example, the fulcrum 161 of the second support 16 and the light bulb 121 of the light bulb seat 12 may be located on the same longitudinal axis.

The rotatable cover 2 is a hollow body adapted to cover the base 11 of the lamp holder 1 from above, where the top end of the rotatable cover 12 is configured with a fan 21 and the bottom end thereof is formed with an opening, and the center of the fan 21 is configured with an axial center 211, which may be a circular hole or a groove indented outward from the inside of the rotatable cover 2; the rotatable cover 2 is used to cover the light bulb 12 of the lamp holder 1, first support 14 and second support 16, and the fulcrum 161 of the second support 16 is adapted to hold the axial center 21 of the fan 21, allowing the rotatable cover 2 to be separated from the base 11, and the rotatable cover 2 thus has a rotation mechanism on the lamp holder 1. Furthermore, the side wall of the rotatable cover 2 is configured with a plurality of hollow holes 22, for example, the rotatable cover 2 may be designed to have a variety of patterns by means of hollowing.

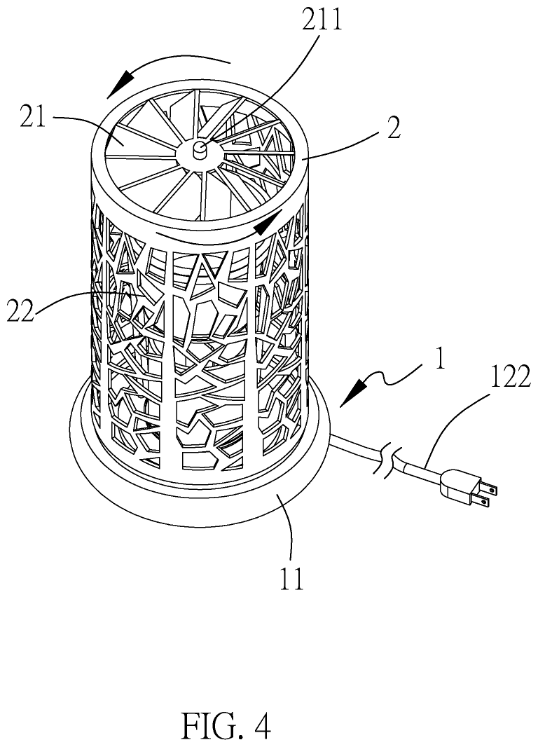

Referring to FIGS. 1 and 4, when the present invention is put into use, the container 14 is filled with essential oil 3, and the bulb 12 is used to heat the essential oil 3 inside the container 15, allowing the essential oil 3 to be vaporized. Furthermore, the heat energy of the bulb 12 can generate rising hot air flow to push the fan 21 configured on the rotatable cover 21 to move, allowing the rotatable cover 2 to be rotated automatically instead of by means of mechanical driving so that the vaporized essential oil can be more spread to indoor space because the rotation of the fan 21 of the rotatable cover 2 increases the flow of airflow.

Furthermore, the plurality of hollow holes 22 configured on the side wall of the rotatable cover 2 allow the light emitted from the light bulb 12 of the lamp holder 1 to penetrate them thereby making the aromatherapy lamp to be more eye-appealing by means of the automatic rotation of the rotatable cover 2 and the light of the light bulb 12 passing through the plurality of hollow holes 22.

I claim:

1. A rotatable aromatherapy lamp, comprising:
a lamp holder, comprising a base, first support and second support, a top face of said base configured with at least one light bulb seat in combination with at least one light bulb configured on a top face of said base, said first support configured with a placement portion in combination with a container and positioned relatively above said light bulb, and said second support configured on said top face of said base and having a fulcrum positioned relatively above said container of said first support; and
a rotatable cover, being a hollow body adapted to cover said base of said lamp holder from above, a top end of said rotatable cover configured with a fan and a bottom end thereof formed with an opening, a center of said fan configured with an axial center, said rotatable cover adapted to cover said light bulb seat, first support and second support of said lamp holder, and said fulcrum of said second support adapted to hold said axial center of said fan, allowing said rotatable cover to be separated from said base.

2. The lamp according to claim 1, wherein said light bulb seat is in electric connection with an electric wire adapted to plug in a mains socket.

3. The lamp according to claim 1, wherein said base is configured with a hollow tube-typed diversion shroud positioned around said light bulb seat.

4. The lamp according to claim 1, wherein a plurality of hollow holes are configured on a side wall of said rotatable cover.

5. The lamp according to claim 1, wherein said placement portion is an annular body.

6. The lamp according to claim 1, wherein said fulcrum is a tip-shaped body.

7. The lamp according to claim 1, wherein said axial center is a groove indented outward from an inside of said rotatable cover.

8. The lamp according to claim 1, wherein said axial center is a circular hole.
